(19) United States
(12) Patent Application Publication
(10) Pub. No.: US 2024/0166682 A1
(43) Pub. Date: May 23, 2024

(54) NOVEL COMPOSITIONS AND METHODS FOR RIBOSOMAL SYNTHESIS OF NUCLEOBASE AMINO ACID POLYMERS AND THEIR CONVERSION INTO NUCLEIC ACIDS

(71) Applicant: Nucleostream, Inc., Austin, TX (US)

(72) Inventors: Bryan Allen BISHOP, Austin, TX (US); Maxwell BERRY, Harrisonburg, VA (US)

(21) Appl. No.: 18/380,456

(22) Filed: Oct. 16, 2023

Related U.S. Application Data
(60) Provisional application No. 63/423,951, filed on Nov. 9, 2022.

(51) Int. Cl. C07H 21/00

(52) U.S. Cl. CPC C07H 21/00

(57) ABSTRACT

Nucleobase amino acid polymer compositions and methods for synthesizing nucleobase amino acid polymers and their conversion to nucleic acids are provided. The nucleobase amino acid polymer compositions disclosed herein comprise nucleobase amino acid monomers that comprise a linker and a nucleobase. The methods disclosed herein comprise synthesizing a nucleic acid polymer by providing a mRNA template, at least one ribosome, at least one nucleobase amino acid tRNA and at least one non-nucleobase amino acid tRNA; and adding a polymerase and at least one primer to the nucleobase amino acid polymer.

### CROSS REFERENCE TO RELATED APPLICATIONS

This application claims the benefit of priority to U.S. Patent application No. 63/423,951, filed Nov. 9, 2022, the contents of which are incorporated herein by reference in its entirety.

### BACKGROUND

Synthesis of long nucleic acids is crucial to the biotechnology industry. For example, biopharmaceutical companies need to synthesize large genes as part of their drug discovery and diagnostic pipelines. For fields like whole-genome synthesis, there is a need to produce extremely long DNA sequences at a low cost. For example, synthesis of a diploid human genome today at market prices would cost over one billion dollars including assembly. Another application requiring synthesis of long nucleic acids is recoding of existing genomes, be they bacterial, fungal, plant, or animal, which presents untold opportunities to reshape medicine, agriculture, and other technologies. And with increasing awareness of pandemic diseases, the ability to rapidly respond to threats becomes even more relevant.

Traditional DNA synthesis applications are limited by cost, time, and efficiency. All current commercial methods use solid-phase synthesis with phosphoramidite chemistry to generate short polymers of oligonucleotides, up to a practical limit of 100-200 nucleotides. The primary limitation in this technique is that the missed incorporation of even a single nucleotide renders the synthesized sequence useless because as genes are translated into proteins using the triplet codon system, a single missing nucleotide in the sequence means that any amino acids translated by the ribosome after a deletion are incorrect, leading to a non-functional and/or truncated protein.

Even in a DNA synthesis reaction with a 99% rate of incorporating the correct nucleotide at the correct time, at a length of 100 nucleotides only 37% of synthesized molecules will have the full-length, correct sequence. One cannot simply mix several of these oligonucleotides into an assembly reaction (combining several shorter oligos into a longer one) and expect the final product to have 100% identity with the desired sequence. As millions of oligonucleotides are synthesized in parallel in a single reaction, extensive sequencing and assembly reactions are required to synthesize useful DNA of a length greater than 200 nucleotides. Modern methods for phosphoramidite synthesis do not directly address this issue, but instead aim to parallelize the synthesis reactions using, for example, semiconductor chips where each reaction is confined to a nanoelectrode. By flowing one of the base monomers over the entire chip, and electrically deblocking only those reactions that require the specific monomer, many millions of reactions can be performed in parallel. Thus, numbers like “millions of base pairs per second” can be claimed, but the ability to generate long, sequence-verified DNA molecules is still fundamentally handicapped by the above issues.

Moreover, traditional phosphoramidite synthesis of DNA takes time. The reactions can take 100 seconds to several minutes to incorporate a single nucleotide. They must flow in each of the four bases over every reaction in turn, to add an average of one nucleotide to each reaction on a chip. Time is also required to ensure that the deblocking and wash steps occur with sufficiently high efficiency. Miniaturization of the reaction also leads to decreased efficiency as parameters become less tightly controlled.

There are limited alternatives to traditional phosphoramidite DNA synthesis. For example, U.S. Publication No. US20200283756A1 discloses a method employing sequential ligation of very short oligonucleotides into a DNA molecule, but that method still relies on phosphoramidite synthesis of the very short DNA oligos. Enzymatic synthesis methods like those disclosed in U.S. Pat. Nos. 9,695,470B2 or 10,837,040B2 require basewise addition of individual nucleotides, with low efficiency and high time cost, providing only an incremental improvement to the process and still limited in the same ways as phosphoramidite chemistry.

Lower costs, improved speed, and improved fidelity of DNA synthesis would result in cost savings for research and development across the entire pharmaceutical and biotech industry or even wider economy including livestock, agriculture, healthcare, etc. There are some current use cases for very long synthetic DNA molecules, but there are likely uses for synthetic DNA that have not been conceived yet because of the extremely high cost of synthetic DNA and the difficulty in synthesizing long contiguous sequences. We are still at an infancy stage in society's ability to synthesize custom DNA molecules cheaply and efficiently at scale.

Accordingly, there is a need for alternative methods to traditional phosphoramidite synthesis that lower costs, take less time, and are more accurate. Specifically, there is a need for synthesis of long nucleic acids with these attributes.

### SUMMARY

The present disclosure is directed to methods for synthesizing a nucleic acid polymer comprising synthesizing a nucleobase amino acid (NAA) polymer, wherein synthesizing a NAA polymer comprises providing a mRNA template, at least one ribosome, at least one NAA-tRNA, at least one non-NAA-tRNA; and adding a polymerase and a primer to the NAA polymer. In some embodiments, the NAA polymer comprises alternating NAA monomers and non-NAA monomers. In some embodiments, the nucleic acid polymer is selected from the group consisting of DNA or RNA. In some embodiments, the step of providing at least one NAA-tRNA and at least one non-NAA-tRNA comprises flowing said NAA-tRNA and non-NAA-tRNAs in a sequential order to result in a NAA polymer of a desired sequence. In some embodiments, the nucleic acid polymer comprises greater than 300 nucleic acid monomers. In some embodiments, the nucleic acid polymer comprises greater than 500 nucleic acid monomers. In some embodiments, the nucleic acid polymer comprises greater than 1,000 nucleic acid monomers. In some embodiments, the nucleic acid polymer comprises greater than 5,000 nucleic acid monomers. In some embodiments, the nucleic acid polymer comprises greater than 10,000 nucleic acid monomers. In some embodiments, the nucleic acid polymer comprises greater than 20,000 nucleic acid monomers. In some embodiments, the nucleic acid polymer comprises greater than 50,000 nucleic acid monomers.

The present disclosure is also directed to methods for synthesizing a NAA polymer comprising: providing a mRNA template, at least one ribosome, at least one NAA-tRNA, at least one non-NAA-tRNA. In some embodiments, the NAA polymer comprises alternating NAA monomers and non-NAA monomers. In some embodiments, the step providing at least one NAA-tRNA and at least one non-NAA-tRNA comprises flowing said NAA-tRNA and non-NAA-tRNAs in a sequential order to result in a NAA polymer of a desired sequence. In some embodiments, the NAA polymer comprises greater than 300 NAA monomers. In some embodiments, the NAA polymer comprises greater than 500 NAA monomers. In some embodiments, the NAA polymer comprises greater than 1,000 NAA monomers. In some embodiments, the NAA polymer comprises greater than 5,000 NAA monomers. In some embodiments, the NAA polymer comprises greater than 10,000 NAA monomers. In some embodiments, the NAA polymer comprises greater than 20,000 NAA monomers. In some embodiments, the NAA polymer comprises greater than 50,000 NAA monomers.

The present disclosure is also directed to compositions comprising a NAA polymer, wherein the NAA polymer comprises NAA monomers of the structure:

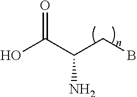

wherein n refers to a linker and B refers to a nucleobase.
In some embodiments, the linker comprises repeating hydrocarbons. In some embodiments, the NAA monomers comprise the structure:

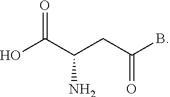

In some embodiments, the NAA monomers are selected from the following structures:

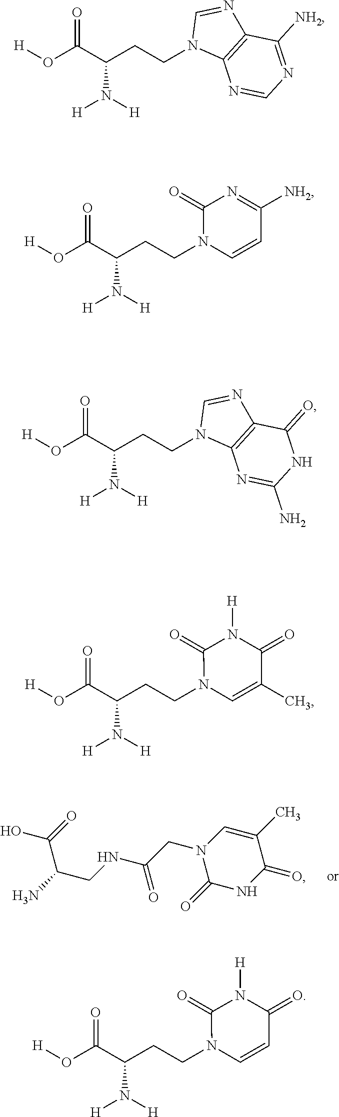 [SMILES: Nc1ncnc2c1ncn2CC[C@H](N)C(=O)O ; Nc1ccn(CC[C@H](N)C(=O)O)c(=O)n1 ; Nc1nc2c(ncn2CC[C@H](N)C(=O)O)c(=O)[nH]1 ; Cc1cn(CC[C@H](N)C(=O)O)c(=O)[nH]c1=O ; Cc1cn(CC(=O)NC[C@H](N)C(=O)O)c(=O)[nH]c1=O ; N[C@@H](CCn1ccc(=O)[nH]c1=O)C(=O)O]

### BRIEF DESCRIPTION OF THE DRAWINGS

FIG. 1 depicts a hydrocarbon linker, with B representing the nucleobase and On representing the number of hydrocarbons.

FIG. 2 depicts a 2-hydrocarbon linker that has been modified with a carbonyl functional group.

FIG. 3A to FIG. 3E depict NAA monomers with 2-carbon linkers with the canonical nucleobases A (FIG. 3A), C (FIG. 3B), G (FIG. 3C), T (FIG. 3D), and U (FIG. 3E).

FIG. 3F depicts a NAA monomer with a 4-atom linker formed by the conjugation of diaminopropionic acid to thymine-1-acetic acid.

FIG. 4 depicts a thymine nucleobase adjacent to glycine. FIG. 5 depicts two thymine nucleobases separated by a serine interstitial amino acid.

FIG. 6A to FIG. 6B depict a PNA poly-thymine base sequence (FIG. 6A) and NAA poly-thymine sequence (FIG. 6B).

### DETAILED DESCRIPTION

The present invention is directed to a method for synthesizing a nucleobase amino acid (“NAA”) polymer comprising providing a mRNA template, at least one ribosome, at least one NAA conjugated to a tRNA, and at least one non-nucleobase amino acid conjugated to a tRNA. The present invention is also directed to a method for synthesizing a nucleic acid polymer from said NAA polymer, comprising adding a polymerase and a primer to the NAA polymer. The present invention is also directed to a composition comprising a NAA polymer, wherein the NAA polymer comprises NAA monomers comprising an amino acid with a nucleobase as its side chain.

As used herein, the singular forms “a”, “an” and “the” include plural referents unless the context clearly dictates otherwise. The use of the term “or” in the claims and the present disclosure is used to mean “and/or” unless explicitly indicated to refer to alternatives only or the alternatives are mutually exclusive.

As used herein, abbreviations for chemical compounds and elements are consistent with their customary use in the art unless otherwise noted. For example, “N” refers to Nitrogen, “C” refers to Carbon, “O ” refers to Oxygen, and “H” refers to Hydrogen.

As used herein, the term “nucleobase amino acid” or “NAA” refers to any amino acid having a nucleobase and linker as its side chain, where the nucleobase is adenine (A), guanine (G), cytosine (C), thymine (T), uracil (U), and derivatives thereof. Non-limiting examples of the derivatives of nucleobases include 5-methylcytosine, 5-hydroxymethylcytosine, 7-deazaguanine, 4-thiouracil, 2-aminopurine, hypoxanthine, and 8-oxoguanine.

As used herein, the term “non-nucleobase amino acid” or “non-NAA” refers to D- or L-alpha amino acids, such as the canonical 20 amino acids, or amino acids with artificial side chains, including but not limited to fluorophore modified amino acids.

As used herein, the term “nucleic acid” refers to DNA, RNA, or any nucleic acid analogue, natural or unnatural (e.g., xeno nucleic acids or XNAs).

As used herein, the term “NAA polymer” refers to a polymer made up of at least 10 contiguous or non-contiguous NAA monomers.

Ribosomes synthesize proteins made up of thousands of amino acid monomers. A eukaryotic ribosome can incorporate approximately 2.5 amino acid monomers into a growing polypeptide per second while prokaryotic ribosomes can incorporate up to 30 amino acid monomers into a growing polypeptide per second. This is much faster than phosphoramidite synthesis of DNA, which incorporates only 1 nucleotide approximately every 100 seconds. Even with this high incorporation rate, ribosomes have very high specificity of incorporation in an environment of multiple competing tRNAs. In the case of a rare codon in mRNA, a ribosome may try several hundred incorrect tRNAs before incorporating the correct one. In fact, a ribosome has an error rate of approximately 10⁻⁵ (1 in 100,000) errors per incorporation, which exceeds the accuracy of phosphoramidite synthesis (10⁻² or 1 in 100 errors per incorporation) by several orders of magnitude. Higher accuracy allows for longer sequences. The ribosome regularly translates proteins comprising thousands of monomers, such as the human muscle protein titin which is composed of approximately 35,000 amino acids.

The ribosome's ability to incorporate monomers other than naturally-occurring amino acids into a growing polypeptide chain is known. See, e.g., WO2022173627A2. This can be accomplished by charging tRNAs with alternative compounds, such as non-naturally occurring amino acids, PNAs, or NAAs. For example, U.S. Pat. No. 10,711,273B2 discloses incorporation of a single NAA into a growing polypeptide chain. However, the end goal of such applications is the polymer synthesized by the ribosomes; no prior studies have attempted to rationally design a polymer comprising several NAAs to generate a desired sequence by a ribosome that could be converted to a nucleic acid by a polymerase.

### NAAs

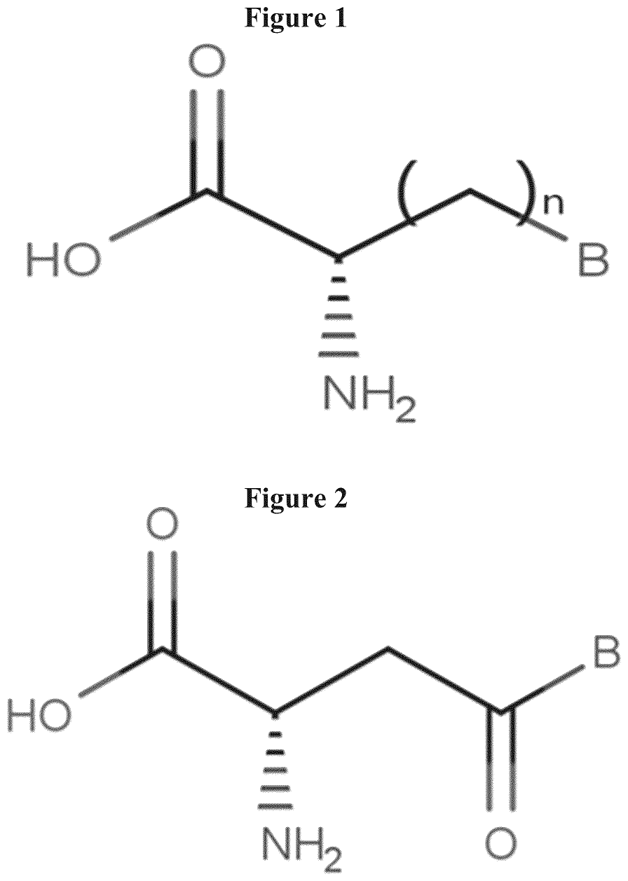

The NAAs of the present invention comprise an amino acid backbone linked to a nucleobase. The linker is preferentially composed of a hydrocarbon backbone, but may be substituted with nitrogen, carbon, oxygen, or any other compatible atom and/or functional group at any or all positions. The backbone may additionally contain other atoms or functional groups bound to the linker, for example a carbonyl, amino, or methyl group. FIG. 1 depicts a hydrocarbon backbone linker, with B representing the nucleobase and On representing the number of hydrocarbons. The linker between the amino acid backbone and the nucleobase may comprise 1-6 atoms, preferably 2-4, and most preferably 2. Any combination of atoms in the linker is possible as long as the resulting NAA is a substrate for incorporation by a ribosome. FIG. 2 depicts a 2-hydrocarbon backbone linker that has been modified with a carbonyl functional group.

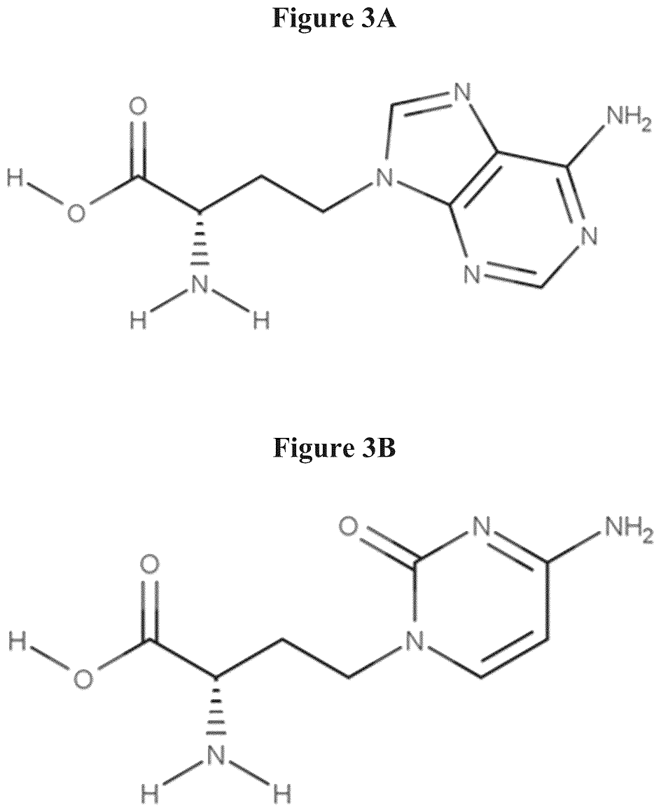

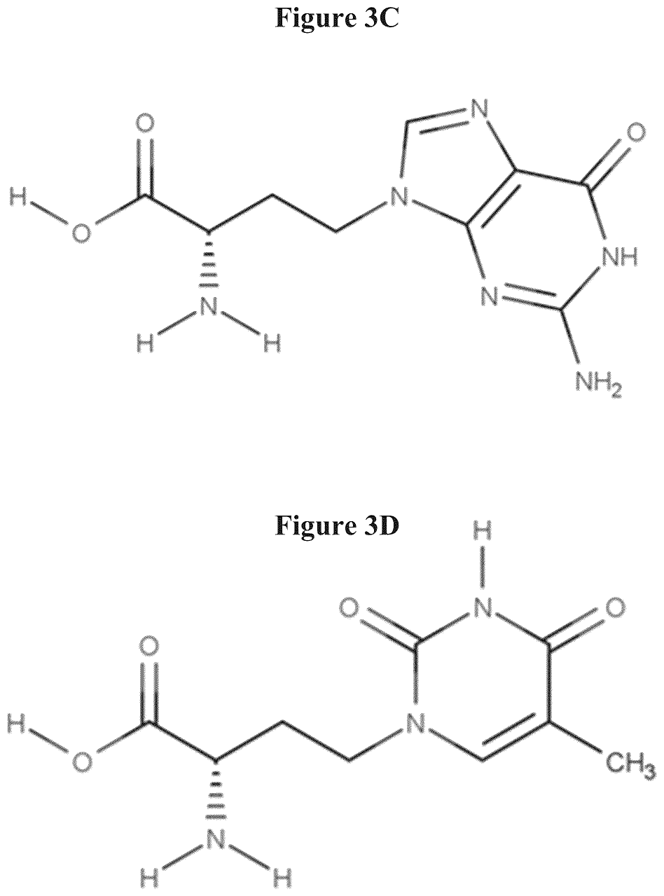

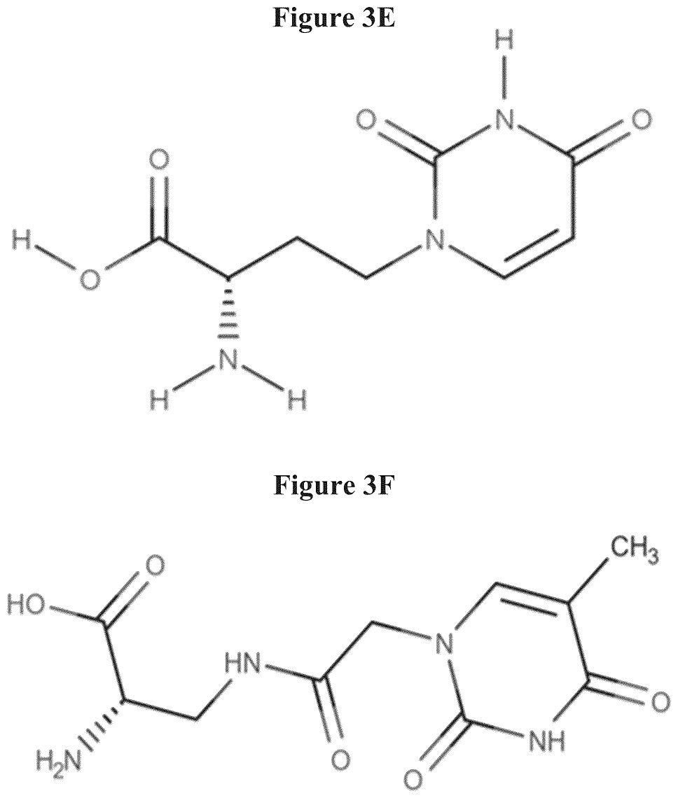

The nucleobase of the NAA may comprise any naturally occurring or engineered nitrogen-containing compound that forms the nucleoside component of a nucleic acid. Naturally occurring, “canonical,” nucleobases comprise adenine (A), cytosine (C), guanine (G), thymine (T) and uracil (U). FIG. 3A to FIG. 3E depict NAA monomers with a 2-carbon linker with the canonical nucleobases A (FIG. 3A), C (FIG. 3B), G (FIG. 3C), T (FIG. 3D), and U (FIG. 3E). FIG. 3F depicts a NAA monomer with a 4-atom linker formed by the conjugation of diaminopropionic acid to thymine-1-acetic acid. Examples of engineered nucleobases for use with the present invention include, but are not limited to nucleobases with fluorescent analogues or other non-canonical bases known in the art. In some embodiments, the NAA polymer is a template for copying by a polymerase. In some embodiments, the NAA polymer is useful as an aptamer.

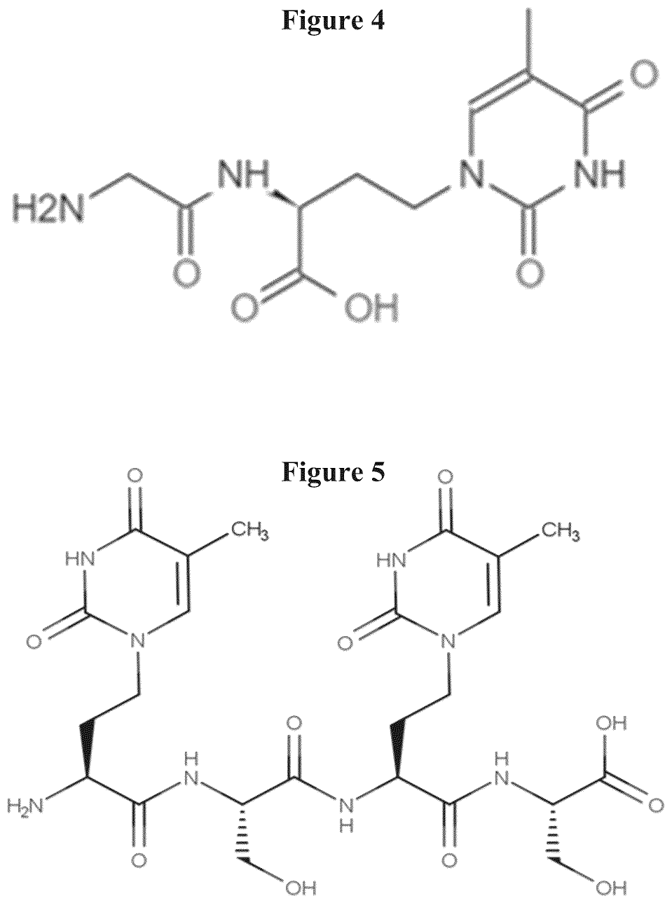

In some embodiments, the NAA monomers are separated by one or more non-NAA monomers. FIG. 4 and FIG. 5 provide illustrative examples of this, FIG. 4 depicting a T nucleobase adjacent to glycine and FIG. 5 depicting two T nucleobases separated by a serine interstitial amino acid. In some embodiments, the non-NAA monomers are small and hydrophilic, for example, serine. In some embodiments, the non-NAA monomers are selected based on effect on backbone charge, solubility in aqueous solutions, hybridization to DNA or RNA, and/or compatibility with a polymerase.

### mRNA

mRNA templates for use herein can be generated through methods known in the art. By way of example but not limitation, long DNA templates are provided and transcribed into mRNA via a RNA transcriptase. The long DNA templates may be generated by any one of several methods known in the art, e.g., the ligation of shorter segments of a repeating subunit into multimers of varying lengths, which could then be separated on the basis of size, and cloned into a plasmid to allow replication. Alternatively, rolling circle amplification of a circular plasmid containing the sequence of one or more repeating subunits could be utilized, potentially using a primer that possesses at its 5′ end a unique sequence. This would allow generation of DNA of arbitrary length, primarily composed of repeating subunits, and use of a primer or primers with a unique sequence would allow further sequence-specific applications. mRNA templates for use herein may be circular or linear.

In some embodiments, the mRNA template comprises alternating series of codons wherein each codon corresponds to either an anticodon to a natural amino acid tRNA or an anticodon to a NAA-tRNA and results in a desired sequence when selected tRNAs are flowed into the reaction. The mRNAs thus function as “universal templates” with which any sequence of NAAs and non-NAAs can be assembled into a polymer by the ribosome, independent of the composition of the template. The sequence of the resulting polymer can be programmed by selecting from a library of NAA-tRNAs or non-NAA-tRNAs which each bear the same anticodon sequence but vary in which amino acid is attached (or “charged”) onto them. The selected tRNA is then injected or flowed into the reaction and attached to the polymer by the ribosome, with unused tRNAs flowing through the reaction chamber to waste. The mRNA template does not alone determine the sequence of the resulting polymer. The order of the tRNA injections, in combination with the universal template, allows programming of the sequence of the resulting polymer.

In some embodiments, the mRNA template comprises repeating subunits of alternating codons and other patterns of codons, where the first codon matches the anticodon of tRNAs charged with a non-NAA, and the following codon matches the anticodon of tRNAs charged with a NAA. In another embodiment, the mRNA template comprises repeating subunits of alternating codons and other patterns of codons, where the first codon matches the anticodon of tRNAs charged with a NAA, and the following codon matches the anticodon of tRNAs charged with a non-NAA. The alternating codons and patterns of codons may comprise alternating patterns such as [ABABABAB]ₙ and [ABCDABCDABCD]ₙ, wherein A is a codon complementary to a tRNA charged with a particular NAA or non-NAA, B is a codon complementary to a tRNA charged with a particular NAA or non-NAA different than that of A, etc. For example, the repeating mRNA template may comprise [GGA CUU CGU AGU]ₙ, wherein GGA and CGU are codons each complementary to a different glycine-bearing tRNA, CUU is complementary to a set of four different nucleobase amino acid tRNAs (each tRNA bears the same anticodon, but is charged with a different nucleobase amino acid), and AGU is complementary to a different set of four nucleobase amino acid tRNAs. The above sequences are chosen at random for illustration purposes only and are not intended to limit the scope of the invention. The same codon can specify four different nucleobase amino acid tRNAs, depending on which one is flowed in to the reaction. In some embodiments, the mRNA templates comprise a pattern or sequence of codons that has been specifically optimized (whether optimized by hand or optimized by algorithm/computer) to avoid ribosome frameshifting or stalling, e.g., by choosing codons that are more likely to result in correct incorporations or to avoid unwanted secondary structures in the mRNA.

mRNA templates for use herein may encode existing or known proteins, or artificial sequence not coding for a known protein. In some embodiments, the mRNA templates comprise modified bases, backbones, additional functional groups, and/or side chain modifications. In some embodiments, the mRNA templates comprise modified bases that increase or decrease tRNA binding fidelity.

### Ribosome

The ribosomes for use herein may comprise wildtype or mutant ribosomes derived from eukaryotes, prokaryotes, thermophiles, and/or extremophiles. In some embodiments, the ribosomes have subunits tethered together. In some embodiments, the ribosomes are constructs having sidechain or chemical modifications that facilitate synthesis of NAA polymers or immobilization in the reaction chamber.

In some embodiments, the ribosomes for use herein can function in environments with high pressure, flow velocity, and/or high concentration of tRNAs.

The ribosomes for use herein may comprise a single ribosome for synthesis of a NAA polymer. Alternatively, the ribosomes for use herein may comprise more than one ribosome for synthesis of a NAA polymer.

### NAA-tRNA

tRNA charged with a NAA (i.e., “NAA-tRNA”) may be generated using any method known in the art, including but not limited to Flexizyme technology (see Nature Protocols 2011, 6(6), 779-790; doi:10.1038/nprot.2011.331; see also Science 1989, 244(4901), 182-188; doi:10.1126/science.2649980) or tRNA synthetases to catalyze the addition of the NAA onto a tRNA. The tRNAs may be wildtype, mutated, or rationally designed tRNAs.

In some embodiments, tRNA libraries are provided such that for each library, the tRNAs all match the same codon/anticodon pair, but each tRNA in a separated tube or liquid or fluid for storage is purified and has a specific charged amino acid or NAA component or monomer. As a non-limiting example, a full library might consist of two libraries where one library is for an AUC codon and the other library is for a CUG codon (codon sequences provided for illustrative purposes only and not intended to limit the scope of the invention). These libraries can be created for any number or length of codons. In an embodiment wherein the mRNA template comprises [ABCDABCDABCD]ₙ, “A” would be one tRNA library, “B” would be another, etc., such that in the tRNA library any NAA or non-NAA to be incorporated into a NAA polymer would be available for selecting as the next reagent. In addition, these libraries can also have natural amino acids and other modified tRNAs that can be used for the next reaction.

### Polymerase

The polymerase of the present disclosure may be any enzyme, naturally occurring or engineered, that can synthesize a nucleic acid strand using a NAA polymer as a template. In some embodiments, the polymerase synthesizes a strand of DNA using a NAA polymer as a template. In other embodiments, the polymerase synthesizes a strand of RNA using a NAA polymer as a template. One of skill in the art will understand that not every polymerase is likely to be able to synthesize a nucleic acid strand using a NAA polymer as a template. Using methods known in the art, one can identify particular polymerases that can synthesize a nucleic acid using a NAA polymer as a template. Some potential polymerases for use in the present disclosure, by way of example but not limitation, include Taq polymerase, Bst polymerase, Vent and Deep Vent polymerases, RNA polymerase II, T7 RNA polymerase, DNA polymerase theta, and reverse transcriptase, or mutants thereof. In some embodiments of the present disclosure, a polymerase is chosen for particular features, such as speed, fidelity, error correction or lack of error correction, temperature sensitivity, and/or thermostability. In some embodiments, a polymerase is engineered, mutated or evolved for particular features, such as speed, fidelity, error correction, lack of error correction, temperature sensitivity, and/or thermostability, especially as those features relate to its function with NAA polymers as a template.

### NAA Polymer

The NAA polymer of the present disclosure comprises alternating NAA monomers and non-NAA monomers. In an embodiment, the NAA polymer comprises about 10 NAA monomers to 50,000 NAA monomers. In a preferred embodiment, the NAA polymer comprises at least 300 NAA monomers.

### Device

The present disclosure also comprises a device for the controlled flow of tRNAs, wherein the tRNAs are charged with non-NAA or NAAs and the device flows the tRNAs over one or more ribosomes in an order according to a predetermined sequence. In some embodiments the device comprises a computing unit that controls the flow and order of tRNAs. In some embodiments the device contains at least one immobilized ribosome. In some embodiments the device comprises at least one immobilized mRNA. Immobilization of at least one ribosome and at least one mRNA may be achieved by methods known in the art, including but not limited to covalent conjugation to a bead, resin, or surface, or non-covalent conjugation.

### EXAMPLES

The following examples are provided to better illustrate the methods of the present disclosure. These examples are not intended to be limited or to otherwise alter the scope of the compositions or methods disclosed herein.

### Example 1: Synthesis of NAA-tRNA

NAA monomers comprising different nucleobases and linker structures are synthesized according to methods known in the art. By way of example but not limitation, such synthesis methods include those disclosed in J. Org. Chem. 1997, 62, 16, 5441-5450, https://doi.org/10.1021/jo970111p; Mol. BioSyst., 2011, 7, 1073-1080, https://doi.org/10.1039/C0MB00214C; Int J Nanomedicine 2018; 13: 2613-2629; https://doi.org/10.2147%2FIJN.S156381, or a custom synthesis method developed using principles of organic synthesis known in the art.

NAA monomers are attached to tRNAs according to methods known in the art such as the Flexizyme technique. See Nature Protocols 2011, 6(6), 779-790; doi:10.1038/nprot.2011.331.

### Example 2: Ribosomal Synthesis of a NAA Polymer

mRNA is generated through methods known in the art. For example, traditional DNA synthesis is used to generate a template comprising an alternating series of codons where each codon corresponds to either an anticodon on a non-NAA-tRNA or an anticodon on a NAA-tRNA. The DNA sequence is then transcribed into mRNA via methods known in the art, such as an in vitro transcription kit. As an example, in vitro transcription kits are commercially available from New England Biolabs, Thermo Fisher Scientific, Takara Bio, Promega, or other suppliers.

The NAA-tRNAs of Example 1 are combined with the mRNA, non-NAA-tRNAs, one or more ribosomes, and other accessory factors necessary for translation of mRNA. The reaction may include fluorophore amino acids attached to tRNAs.

To show that the ribosome can incorporate NAA-tRNAs into a polymer, the tRNAs do not need to be fed in sequentially, and can all be present at the beginning of the reaction, allowing it to proceed at the speed of the ribosome's catalytic capacity. For purposes of controlled applications where the particular sequence of the NAA polymer is essential, techniques known in the art wherein tRNAs are sequentially flowed over ribosomes may be used.

The resulting NAA polymers will be analyzed to detect yield, length, and accuracy of the NAA polymer sequence. Analysis may include methods known in the art such as (1) mass spectroscopic analysis of the reaction product to determine the size and composition of the NAA polymers produced; (2) quantification of charged and uncharged tRNAs before/after the reaction, to determine utilization of the charged tRNAs by the ribosome, as a proxy for their incorporation into a polymer; (3) addition of fluorophore-labeled DNA or RNA oligonucleotide to detect NAA polymers via Förster resonance energy transfer (FRET) or similar methods, wherein the labeled NAA and DNA/RNA polymers will interact through base-pairing and be detected by FRET; (4) detection of binding to DNA or RNA of the resulting NAA polymer using surface plasmon resonance (Biacore) or similar methods; and (5) other methods known in the art for detection of biomolecules, especially nucleotide-binding molecules.

The ribosomal synthesis reaction may be optimized using various methods known in the art, such as modifying quantities of components, temperature, pH, additives, NAA linker sequence, mRNA sequence, interstitial amino acid type and pattern (eg purely glycine, alternating glycine and serine, or any other combination of any of the natural 20 amino acids or other unnatural amino acids known in the art), or by using variants (naturally-occurring or artificial) of accessory factors (e.g., elongation factors) and tRNA. The ribosomal synthesis reaction may be optimized by performing directed evolution of the proteins, tRNAs, and other components which can have their sequence mutated to increase yields, accuracy, efficiency, and speed of the reaction, by screening variants in parallel, identifying the most useful variants, and optionally performing another round or rounds of directed evolution from the previously mutated sequence. Further, this can be performed in tandem with the development of the NAA polymer as a polymerase template for purposes of facilitating the use of the NAA polymer as a template for a polymerase, i.e. optimization can focus on the backbone and linker structures which are most favorable to the polymerase reaction, and vice versa.

### Example 3: Synthesis of Nucleic Acid From NAA Polymer

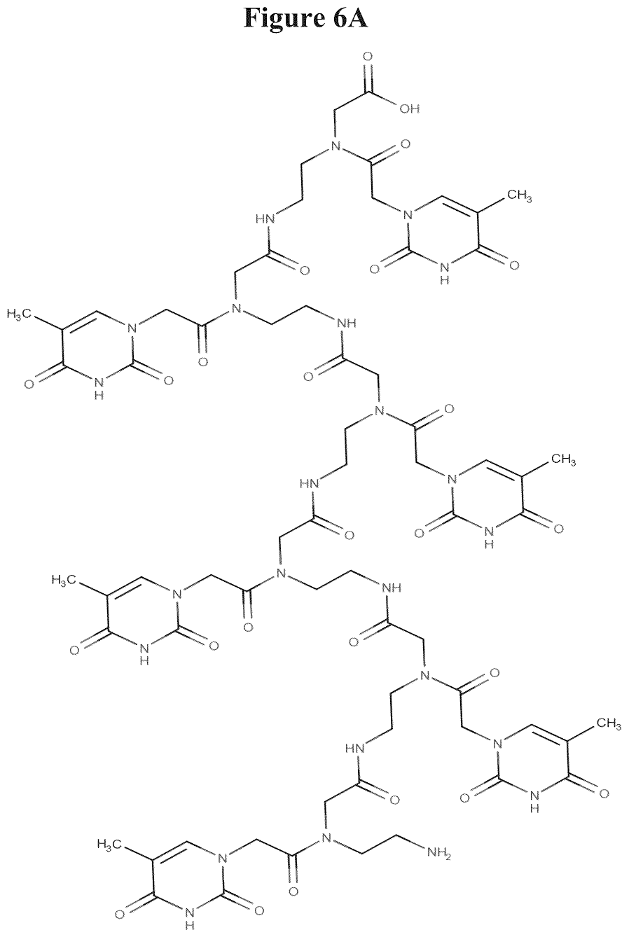

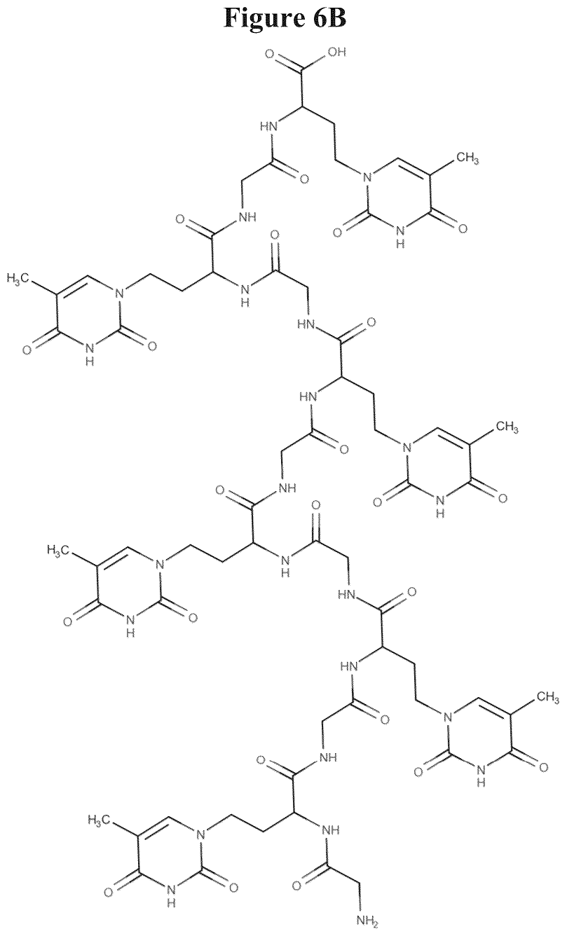

In 2021, the first report of using peptide nucleic acid (PNA) as a template for PCR was reported in Biochem. and Biophys. Res. Comms. 2021, 579, 19, 76-80; doi: 10.1016/j.bbrc.2021.09.057. Given the similarity in structure of PNA polymer to NAA polymer as depicted in FIG. 6A (PNA poly-thymine sequence) and FIG. 6B (NAA p oly-thymine sequence), we expect that NAA polymers can serve as a template for PCR when a complementary primer is added. Previous research with glycol nucleic acids (GNAs) indicates that even XNAs with a weak binding to DNA/RNA can function as a template for a polymerase. See Proc Natl Acad Sci USA. 2007, 104(37), 14598-603; doi:10.1073/pnas.0704211104.

To illustrate this, NAA polymers resulting from the process described in Example 2 or synthesized according to methods known in the art such as solid-phase peptide synthesis will be incubated with DNA or RNA primers, a polymerase, dNTPs or NTPs, and other additives to begin a reaction similar to the common isothermal reactions for DNA amplification or PCR.

After completion of the reaction, expected reaction products (double stranded DNA, double stranded RNA, or DNA:RNA hybrid, or single-stranded DNA or RNA) corresponding to the nucleobase sequence of the NAA polymer or its reverse complement can be detected through methods common in the art. For example, agarose gel electrophoresis to detect the presence and approximate size of polymerized nucleotides, or Sanger sequencing to determine the sequence of the polymerized nucleotides, or a combination thereof, or other methods.

Real-time detection of nucleotide incorporation can also be accomplished through the use of labeled nucleotides (fluorophores conjugated to the terminal phosphate moiety of NTPs or dNTPs) which emit light upon incorporation into a growing strand.

Components and conditions of the reaction will be varied to optimize the reaction, including concentrations of all components, temperature, pH, additives, polymerase type, primer sequence and length, and type of NAA polymer (sequence, length, interstitial amino acid type, linker type). Reactions may proceed isothermally (at a set temperature) or with thermal cycling (as is commonly used in PCR). This includes directed evolution of polymerases through mutation to optimize speed, fidelity, and yield of the reaction.

Experiments can be performed in tandem with the development of the ribosome reaction, i.e. optimization of polymerase conditions can focus on the backbone and linker structures which are most favorable to the ribosome reaction.

What is claimed is:

1. A method for synthesizing a nucleic acid polymer comprising:
synthesizing a NAA polymer, wherein synthesizing a NAA polymer comprises providing a mRNA template, at least one ribosome, at least one NAA-tRNA, and at least one non-NAA-tRNA; and
adding a polymerase and at least one primer to the NAA polymer.

2. The method of claim 1, wherein the NAA polymer comprises alternating NAA monomers and non-NAA monomers.

3. The method of claim 1, wherein the nucleic acid polymer is selected from the group consisting of DNA or RNA.

4. The method of claim 1, wherein the step of providing at least one NAA-tRNA and at least one non-NAA-tRNA comprises flowing said NAA-tRNA and non-NAA-tRNAs in a sequential order to result in a NAA polymer of a desired sequence.

5. The method of claim 1, wherein the nucleic acid polymer comprises greater than 300 nucleic acid monomers.

6. The method of claim 1, wherein the nucleic acid polymer comprises greater than 500 nucleic acid monomers.

7. The method of claim 1, wherein the nucleic acid polymer comprises greater than 1,000 nucleic acid monomers.

8. The method of claim 1, wherein the nucleic acid polymer comprises greater than 5,000 nucleic acid monomers.

9. The method of claim 1, wherein the nucleic acid polymer comprises greater than 10,000 nucleic acid monomers.

10. The method of claim 1, wherein the nucleic acid polymer comprises greater than 20,000 nucleic acid monomers.

11. The method of claim 1, wherein the nucleic acid polymer comprises greater than 50,000 nucleic acid monomers.

12. A method for synthesizing a NAA polymer comprising: providing a mRNA template, at least one ribosome, at least one NAA-tRNA, and at least one non-NAA-tRNA.

13. The method of claim 12, wherein the NAA polymer comprises alternating NAA monomers and non-NAA monomers.

14. The method of claim 12, wherein the step of providing at least one NAA-tRNA and at least one non-NAA-tRNA comprises flowing said NAA-tRNA and non-NAA-tRNAs in a sequential order to result in a NAA polymer of a desired sequence.

15. The method of claim 1, wherein the NAA polymer comprises greater than 300 NAA monomers.

16. The method of claim 1, wherein the NAA polymer comprises greater than 500 NAA monomers.

17. The method of claim 1, wherein the NAA polymer comprises greater than 1,000 NAA monomers.

18. The method of claim 1, wherein the NAA polymer comprises greater than 5,000 NAA monomers.

19. The method of claim 1, wherein the NAA polymer comprises greater than 10,000 NAA monomers.

20. The method of claim 1, wherein the NAA polymer comprises greater than 20,000 NAA monomers.

21. The method of claim 1, wherein the NAA polymer comprises greater than 50,000 NAA monomers.

22. A composition comprising a NAA polymer, wherein the NAA polymer comprises NAA monomers of the structure: 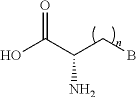
wherein n refers to a linker and B refers to a nucleobase.

23. The composition of claim 22, wherein the linker comprises repeating hydrocarbons.

24. The composition of claim 22, wherein the NAA monomers comprise the structure:

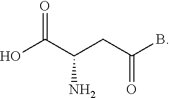

25. The composition of claim 22, wherein the NAA monomers are selected from the following structures: 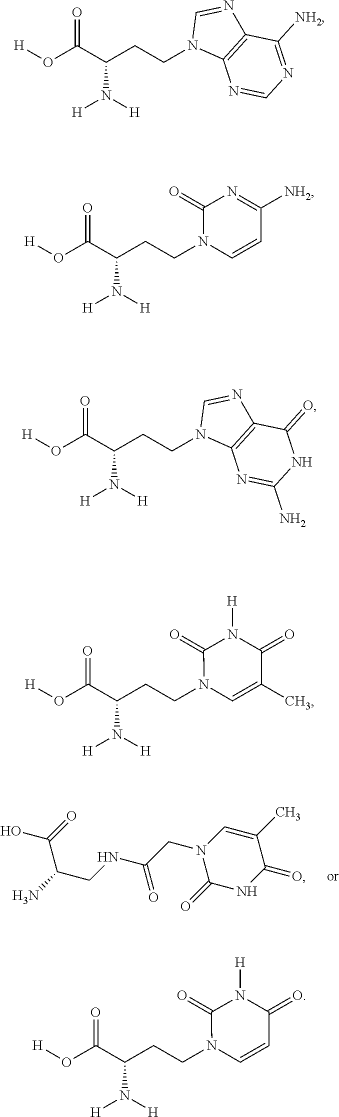 [SMILES: Nc1ncnc2c1ncn2CC[C@H](N)C(=O)O ; Nc1ccn(CC[C@H](N)C(=O)O)c(=O)n1 ; Nc1nc2c(ncn2CC[C@H](N)C(=O)O)c(=O)[nH]1 ; Cc1cn(CC[C@H](N)C(=O)O)c(=O)[nH]c1=O ; Cc1cn(CC(=O)NC[C@H](N)C(=O)O)c(=O)[nH]c1=O ; N[C@@H](CCn1ccc(=O)[nH]c1=O)C(=O)O]
